(19) United States
(12) Patent Application Publication
(10) Pub. No.: US 2025/0155351 A1
(43) Pub. Date: May 15, 2025

(54) OPTICAL MEASUREMENT APPARATUS AND OPTICAL MEASUREMENT METHOD

(71) Applicant: SAMSUNG ELECTRONICS CO., LTD., Suwon-si (KR)

(72) Inventors: Deokhyeon Kwon, Suwon-si (KR); Azer YALIN, Fort Collins, CO (US)

(73) Assignees: SAMSUNG ELECTRONICS CO., LTD., Suwon-si (KR); Colorado State University Research Foundation (CSURF), Fort Collins, CO (US)

(21) Appl. No.: 18/947,891

(22) Filed: Nov. 14, 2024

Related U.S. Application Data
(60) Provisional application No. 63/548,462, filed on Nov. 14, 2023.

(30) Foreign Application Priority Data
Jan. 5, 2024 (KR) 10-2024-0002336
Feb. 28, 2024 (KR) 10-2024-0029232

(51) Int. Cl. G01N 15/075; G01N 33/00

(52) U.S. Cl. CPC G01N 15/075; G01N 33/0027

(57) ABSTRACT

An optical measurement method includes emitting light to an optical beam path cell including a first mirror and a second mirror that faces the first mirror, forming an optical beam path by reflecting the light between the first mirror and the second mirror, obtaining an optical signal including an optical characteristic value based on an interaction of the light with a sample in the optical beam path, separating the optical signal into a gas signal and a particle signal, and determining a concentration of particles in the sample based on the particle signal, where the determining of the concentration of the particles includes fitting the particle signal into a first distribution function indicating the optical characteristic value and a frequency of the optical characteristic value, and the concentration of particles is determined based o999n first shape information about the first distribution function.

### CROSS-REFERENCE TO RELATED APPLICATIONS

This application is based on and claims priority under 35 U.S.C. § 119 to U.S. Patent Application No. 63/548,462, filed on Nov. 14, 2023, in the U.S. Patent & Trademark Office, Korean Patent Application No. 10-2024-0002336, filed on Jan. 5, 2024, in the Korean Intellectual Property Office, and Korean Patent Application No. 10-2024-0029232, filed on Feb. 28, 2024, in the Korean Intellectual Property Office, the disclosures of which are incorporated by reference herein in their entireties.

### BACKGROUND

Example embodiments of the disclosure relate to an optical measurement device and an optical measurement method.

Spectrometers may be used to measure the characteristics and concentration of a sample being analyzed through a phenomenon in which light reacts with a material and is scattered or absorbed. Among these spectrometers, open path spectrometers may be used, as open path spectrometers have a physical sampling space that is open to the atmosphere in order to minimize a phenomenon in which a sample being analyzed is adsorbed onto a flow path. An open path spectrometer may measure a gas concentration by using a mixed signal of condensed particles and gas molecules in the atmosphere.

Information disclosed in this Background section has already been known to or derived by the inventors before or during the process of achieving the embodiments of the present application, or is technical information acquired in the process of achieving the embodiments. Therefore, it may contain information that does not form the prior art that is already known to the public.

### SUMMARY

One or more example embodiments provide an optical measurement device and an optical measurement method that may be capable of determining both the concentration of gas and the concentration of particles from a spectroscopic signal.

Additional aspects will be set forth in part in the description which follows and, in part, will be apparent from the description, or may be learned by practice of the presented embodiments.

According to an aspect of an example embodiment, an optical measurement method may include emitting light to an optical beam path cell including a first mirror and a second mirror that faces the first mirror, forming an optical beam path by reflecting the light between the first mirror and the second mirror, obtaining an optical signal including an optical characteristic value based on an interaction of the light with a sample in the optical beam path, separating the optical signal into a gas signal and a particle signal, and determining a concentration of particles in the sample based on the particle signal, where the determining of the concentration of the particles includes fitting the particle signal into a first distribution function indicating the optical characteristic value and a frequency of the optical characteristic value, and the concentration of particles is determined based on first shape information about the first distribution function.

According to an aspect of an example embodiment, an optical measurement device may include a light source configured to emit light, an optical beam path cell including a first mirror and a second mirror configured to form an optical beam path by reflecting the light emitted from the light source, and a detector configured to obtain an optical signal based on an interaction of the light with an aerosol sample in the optical beam path, and determine a concentration of particles and a concentration of gas in the aerosol sample from the optical signal, where the detector includes a concentration determination module configured to fit a particle signal extracted from the optical signal into a Gaussian distribution function representing a ring-down time value and a frequency of the ring-down time value, and determine the concentration of the particles based on first shape information about the Gaussian distribution function.

According to an aspect of an example embodiment, an optical measurement method may include emitting light to an optical beam path cell including a first mirror and a second mirror that faces the first mirror, forming an optical beam path by reflecting the light between the first mirror and the second mirror, measuring an optical signal from an aerosol sample in the optical beam path, separating the optical signal into a gas signal and a particle signal, and determining a concentration of particles and a concentration of gas in the aerosol sample based on the particle signal, where the determining of the concentration of the particles includes fitting the particle signal into a Gaussian distribution function indicating a ring-down time value and a frequency of the ring-down time value, and the concentration of the particles is determined based on a mean, a variance, a skewness, and a kurtosis of the Gaussian distribution function.

### BRIEF DESCRIPTION OF DRAWINGS

This application file contains at least one drawing executed in color. Copies of this application publication with color drawing(s) will be provided by the Office upon request and payment of the necessary fee.

The above and other aspects, features, and advantages of certain example embodiments of the present disclosure will be more apparent from the following description taken in conjunction with the accompanying drawings, in which:

FIG. 1 is a flowchart illustrating an optical measurement method according to one or more embodiments;

FIG. 2 is a diagram illustrating an optical measurement device according to one or more embodiments;

FIG. 3 is a graph illustrating intensity of light measured by the optical measurement device of FIG. 2 over time, according to one or more embodiments;

FIG. 4 is a flowchart illustrating an operation of determining a concentration of particles in an optical measurement method according to one or more embodiments;

FIG. 5 is a graph illustrating an optical signal including an optical characteristic value obtained by an optical measurement device according to one or more embodiments;

FIG. 6 is a graph illustrating a result of fitting a particle signal to a first distribution function indicating a frequency of an optical characteristic value in an optical measurement method according to one or more embodiments;

FIGS. 7A to 7C are graphs illustrating secondary to quaternary standard moments of a first distribution function extracted in an optical measurement method according to one or more embodiments; and

FIG. 8 is a graph illustrating a result of fitting a particle signal to a second distribution function indicating a cumulative frequency of an optical characteristic value in an optical measurement method according to one or more embodiments.

### DETAILED DESCRIPTION

Hereinafter, example embodiments of the disclosure will be described in detail with reference to the accompanying drawings. The same reference numerals are used for the same components in the drawings, and redundant descriptions thereof will be omitted. The embodiments described herein are example embodiments, and thus, the disclosure is not limited thereto and may be realized in various other forms.

As used herein, expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

It will be understood that when an element or layer is referred to as being “over,” “above,” “on,” “below,” “under,” “beneath,” “connected to” or “coupled to” another element or layer, it can be directly over, above, on, below, under, beneath, connected or coupled to the other element or layer or intervening elements or layers may be present. In contrast, when an element is referred to as being “directly over,” “directly above,” “directly on,” “directly below,” “directly under,” “directly beneath,” “directly connected to” or “directly coupled to” another element or layer, there are no intervening elements or layers present.

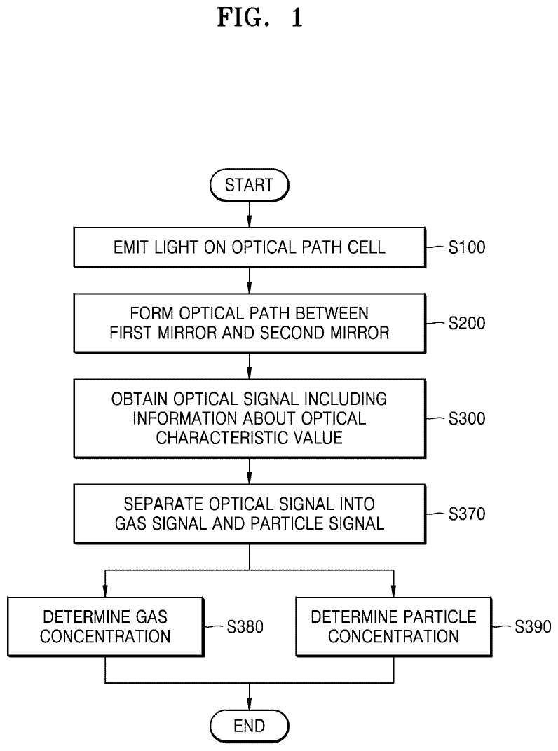

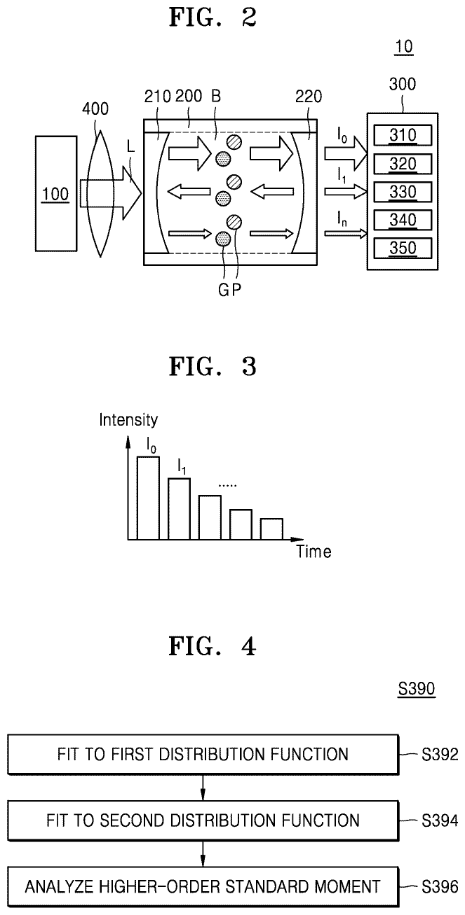

FIG. 1 is a flowchart illustrating an optical measurement method according to one or more embodiments. FIG. 2 is a diagram illustrating an optical measurement device according to one or more embodiments. FIG. 3 is a graph illustrating intensity of light measured by the optical measurement device of FIG. 2 over time, according to one or more embodiments.

Referring to FIGS. 1 and 2, the optical measurement method according to one or more embodiments may include operation S100 of emitting light L to an optical beam path cell 200, operation S200 of forming an optical beam path B by reflecting the light L between a first mirror 210 and a second mirror 220, operation S300 of obtaining an optical signal including an optical characteristic value, operation S370 of separating the optical signal into a gas signal and a particle signal, operation S380 of determining a concentration of gas, and operation S390 of determining a concentration of particles.

Referring to FIG. 2, the optical measurement device 10 may include a light source 100 for generating light, an optical beam path cell 200 having a first mirror 210 and a second mirror 220 placed opposite to each other and reflecting incident light to generate an optical beam path B, and a detector 300 for measuring an optical signal from a sample present in the optical beam path B. In addition, the optical measurement device 10 may further include a lens 400.

In one or more embodiments, the optical measurement device 10 may refer to an analysis device for identifying the relationship between physical information such as the concentration of materials and the optical properties of the materials by using a light extinction reaction to the materials present inside an optical beam path B. Light may be scattered and absorbed inside the optical measurement device 10 to be extinguished.

The optical measurement device 10 may include a cavity ring-down spectrometer (CRDS). The CRDS may refer to a spectrometer that uses a phenomenon in which some of light that is generated from a light source and that passes through various optical components is introduced into a space aligned to form a Fabry-Perot Interferometer structure where two Plano-concave high reflectivity (HR) mirrors of the same shape with one surface flat and the other surface curved are on both sides facing each other, and are reflected and extinguished by materials existing between the two mirrors.

The optical measurement device 10 may analyze a sample present inside the optical beam path B. The sample may be an aerosol (a collection of solid or liquid particles suspended in a gas), and may include gas and liquid droplets or solid particles.

In one or more embodiments, the optical beam path B of the optical beam path cell 200 may have an open path form that is exposed to the environment. When the optical measurement device 10 is in the form of an open path, the optical measurement device 10 may be used in fields such as atmospheric science, gas and particle pollution measurement in clean room air, where the need for the optical beam path B to be exposed to the environment is recognized.

When the optical beam path B has an open path form, the reaction speed of the material (aerosol) present inside the optical beam path B and the light L may be faster than when the optical beam path has a closed path form. In addition, when the optical beam path B has an open path form, there may be relatively more particles inside the optical beam path B than when the optical beam path has a closed path form.

When the optical measurement device 10 is in the open path form, data on a gaseous sample and a solid sample present in an aerosol may be simultaneously measured by a detector 300. Data on gaseous and solid samples may be effectively separated using algebraic and statistical methodologies.

In one or more embodiments, the optical measurement device 10 may have a closed path form in which the optical beam path B is cut off from the environment (e.g., the outside environment). When the optical measurement device 10 has a closed path form, the optical measurement device 10 may further include a blocking member. The gas inside the optical measurement device 10 may be disconnected from the external gas by the blocking member. Gas containing impurities to be measured may be supplied inside the blocking member. A pressure regulator connected to the blocking member may maintain a constant pressure in the blocking member.

In one or more embodiments, the optical measurement device 10 may be used in spectroscopy technology based on the Beer-Lambert law. For example, the optical measurement device 10 may use single pass spectroscopy that emits light in only one direction. For example, the single pass spectroscopy may include ultraviolet-visible (UV-vis) spectroscopy, Fourier transformed infrared (FT-IR) spectroscopy, near infrared and far-infrared (NIR and far-IR) spectroscopy, terahertz (THz) spectroscopy, sub-millimeter (sub-mm) spectroscopy, etc.

In one or more embodiments, the optical measurement device 10 may use multi-pass spectroscopy using the first mirror 210 and the second mirror 220. For example, the multi-pass spectroscopy may include Pfund cell, White cell, Herriott cell, Fabry-Perot Etalon/Resonator/Interferometer, etc. The multi-pass spectroscopy may include CRDS, integrated cavity output spectroscopy (ICOS), cavity enhanced absorption spectroscopy (CEAS), cavity attenuated phase shift (CAPS) spectroscopy, etc.

In one or more embodiments, the optical measurement device 10 may include a combination of photoacoustic spectroscopy (PAS), quartz-enhanced PAS, single pass spectroscopy, and multi-pass spectroscopy in various forms.

Referring to FIGS. 1 and 2, the light source 100 may emit the light L into the optical beam path cell 200 having the first mirror 210 and the second mirror 220 in operation S100. In one or more embodiments, the light source 100 may generate light L of a preset wavelength according to the type of particles to be measured. For example, the light L of a preset wavelength may include at least one of UV, visible, mid-IR, near-IR, far-IR, sub-mm, and THz. The light source 100 may generate light L having a preset frequency by being selected according to the type of gas G and particles P included in an aerosol to be measured.

The light source 100 may emit the light L to the first mirror 210 located adjacent to the light source 100, and the light L may pass through the first mirror 210 to the second mirror 220. Alternatively, the light source 100 may emit the light L to the lens 400, and the light L may be collected and focused by the lens 400. The lens 400 may include a component that adjusts the shape and intensity distribution of the light L, a component that removes reverse-reflected light and induces unwanted measurement results, and the like.

The light source 100 may stop generating additional light through feedback with the detector 300 while the light L is reflected and extinguished by being emitted to the first and second mirrors 210 and 220. Therefore, overlapping of optical signal analysis data by additional light may be prevented. When optical signal analysis data is determined by the detector 300, the light source 100 may receive a measurement completion signal from the detector 300 and resume generation of light.

Referring to FIGS. 1 and 2, the first and second mirrors 210 and 220 of the optical beam path cell 200 may be disposed to face each other to reflect light incident from the light source 100 to form an optical beam path B in operation S200. The first and second mirrors 210 and 220 may be highly reflective mirrors. The first and second mirrors 210 and 220 may include two plano-concave HR mirrors having the same shape with one surface flat and the other curved. The first and second mirrors 210 and 220 may include a Fabry-Perot interferometer structure installed to face each other.

The light L may be continuously reflected between the first and second mirrors 210 and 220. The light L may be partially extinguished by reflecting and colliding between the first and second mirrors 210 and 220. The light L may be scattered and absorbed between the first and second mirrors 210 and 220 to be extinguished. Each time the light L is reflected by the first and second mirrors 210 and 220, the light L may be extinguished, so that the intensity thereof may be weakened.

The light L may be partially extinguished while reflecting and colliding with the gas G and the particles P present inside the optical beam path B. Each time the light L collides with the gas G and the particles P, the light L may be extinguished, and thus the intensity thereof may be weakened. The light L may pass through the second mirror 220 and be incident on the detector 300.

Referring to FIGS. 1 and 2, the detector 300 may obtain an optical signal in operation S300. The optical signal may correspond to an expression including an optical signal and an electrical signal in which the optical signal is converted. The optical signal may include an optical characteristic value due to an interaction between the light L and a sample (e.g., an aerosol) present in an optical beam path. In one or more embodiments, the optical characteristic value may be a ring-down time value, and a detailed description will be described later.

In one or more embodiments, the detector 300 may include an optical signal detection module 310, an electrical signal conversion module 320, an electrical signal recording module 330, a concentration determination module 340, and a memory 350. The term “module” may refer a functional and structural combination of hardware for performing operations described herein and software for driving the hardware. For example, the “module” may refer a logical unit of a predetermined code and a hardware resource for performing the predetermined code, and does not necessarily refer a physically connected code or a type of hardware. The memory 350 may include volatile and/or nonvolatile memory, such as dynamic random access memory (RAM) (DRAM), static RAM (RAM), read-only memory (ROM), and variations thereof for storing instructions to be executed by a processor (e.g., the optical signal detection module 310, the electrical signal conversion module 320, the electrical signal recording module 330, the concentration determination module 340, etc.). The memory 350 may also store other data, and may be implemented in a device separate from the detector 300.

As shown in FIGS. 2 and 3, the optical signal detection module 310 may separate the light transmitted through the second mirror 220 according to the number of times the light has been reflected and receive the separated light as input. The intensity of light may be exponentially decayed in the form of an exponential function over time. The intensity of light may be classified into unreflected light I₀, one-time reflected light I₁, n-time reflected light Iₙ, and the like according to the degree of decay. For example, the one-time reflected light I₁ may refer to light transmitted through the second mirror 220 after the light reflected from the second mirror 220 is reflected from the first mirror 210. When the number of reflections increases, the intensity of the light may decrease. The optical signal detection module 310 may receive, as an input, light having intensity which decreases over time. When the intensity of light decreases to less than or equal to a preset intensity, the optical signal detection module 310 may stop measuring the light and transmit a measurement completion signal to the light source 100 such that the light source 100 may emit new light.

The electrical signal conversion module 320 may convert light received as input from the optical signal detection module 310 into an electrical signal. The electrical signal recording module 330 may record the intensity of the electrical signal over time. The concentration determination module 340 may determine the concentration of the gas G and the concentration of the particles P by processing the data recorded by the electrical signal recording module 330.

The concentration determination module 340 may analyze the optical signal obtained by the detector 300 through a mathematical relationship based on the Beer-Lambert law by using the extinction of the intensity of light. For example, the mathematical relational equation may include a mathematical relationship showing the relationship between the measured values of several multi-pass spectroscopy such as CRDS, CEAS, and CAPS and the optical properties of the material (e.g., an extinction coefficient). The concentration determination module 340 may analyze the mathematical relationship and express the mathematical relationship as an extinction coefficient (αext), which may be the sum of optical characteristic reactions of the materials G and P in the optical beam path.

The concentration determination module 340 may classify the extinction coefficient according to the step in which the intensity of light is extinguished in the form of an exponential function. The concentration determination module 340 may express an extinction coefficient using an optical cross-section (σext), which is an inherent characteristic of a material. The concentration determination module 340 may express the extinction coefficient as a product of an optical cross-sectional area and a concentration (N) of aerosol according to an extinguishing step. The concentration determination module 340 may express the extinction coefficient as in Equation (1) below.

$${α}_{ext} = \sum {σ}_{ext} \cdot N \qquad ( 1 )$$

In Equation (1), αₑₓₜ is the extinction coefficient, σₑₓₜ is an optical cross-sectional area, and N is a concentration of aerosol.

In the case of CRDS, the extinction coefficient may be expressed using the difference in ring-down time as in Equation (2).

$${α}_{e x t} \frac{d}{L} = \frac{1}{c} ( \frac{1}{τ} - \frac{1}{{τ}_{0}} ) \qquad ( 2 )$$

In Equation (2), αₑₓₜ is the extinction coefficient, d is the distance between an inlet and an outlet, L is the distance between mirrors, c is the speed of light, τ is the ring-down time value when a material is present in the cavity, and to is the ring-down time value in the empty cavity.

In the case of ICOS or CEAS, the extinction coefficient may be expressed as in Equation (4) through the assumption of Equation (3) that the sum of the light intensities is proportional to the ring-down time value.

$$τ ∝ {\int }_{0}^{∞} Idt \qquad ( 3 )$$

$$\frac{{I}_{0} - I}{I} = \frac{{α}_{e x t} d}{1 - R} \qquad ( 4 )$$

In Equations (3) and (4), αₑₓₜ is the extinction coefficient, d is the distance of the inlet and the outlet, R is the radius of curvature of the mirror, τ is the ring-down time value, and I is the intensity of light.

In the case of CAPS, the extinction coefficient may be expressed using the phase shift of light, as in Equations (5) and (6).

$$tan [ ϕ ( v ) ] = - ω τ ( v ) \qquad ( 5 )$$

$${α}_{ext} \frac{d}{L} = \frac{ω}{c} ( \frac{1}{tan ϕ ( v )} - \frac{1}{tan {ϕ}_{0} ( v )} ) \qquad ( 6 )$$

In Equations (5) and (6), αₑₓₜ is the extinction coefficient, d is the distance of the inlet and the outlet, L is the distance between mirrors, c is the speed of light, ω is the frequency, and τ is the ring-down time value.

In this way, even when using ICOS, CEAS, or CAPS as well as CRDS, substantially the same data may be obtained.

The concentration determination module 340 may simultaneously analyze the signal of the particle P and the signal of the gas G in the optical beam path B. The concentration determination module 340 may separate the optical signal into a gas signal and a particle signal from an optical signal including an optical signal and/or an electrical signal in operation S370. The concentration determination module 340 may effectively separate the simultaneously measured optical signals into particle signals and gas signals using algebra and statistical methodologies.

Specifically, the concentration determination module 340 may represent optical signals detected, converted, and/or recorded through the optical signal detection module 310, the electrical signal conversion module 320, and/or the electrical signal recording module 330 as a distribution graph of optical characteristic values. In one or more embodiments, the optical characteristic value may be a value representing the degree of extinction of the light L. In one or more embodiments, the optical characteristic value may be a ring-down time (τ) value, and the concentration determination module 340 may determine a graph representing the ring-down time value and the frequency of the ring-down time value (frequency of data having a corresponding optical characteristic value).

In general, compared to the case of a sample made of only gas, a ring-down time value may be shifted in the case of a sample containing particles. In one or more embodiments, the concentration determination module 340 may separate the optical signal into a gas signal and a particle signal by using the shift phenomenon of the statistically obtained ring-down time value. Since the gas signal and the particle signal separated from the optical signal are combined with each other, they may be expressed as Equation (7).

$${α}_{ext} = \sum {σ}_{ext} \cdot N = \sum ( {σ}_{ext , particle} \cdot {N}_{particle} + {σ}_{ext , gas} \cdot {N}_{gas} ) \qquad ( 7 )$$

In Equation (7), αext is the extinction coefficient, σext is the optical cross-sectional area, N is the concentration of aerosol, σext,particle is the optical property of solid or liquid particles, Nparticle is the concentration of solid or liquid particles, σext,gas is the optical property of gas, and Ngas is the concentration of gas.

With respect to determination of the gas signal and the gas concentration, the concentration determination module 340 may determine the gas concentration by converting the separated gas signal into a gas concentration using the optical characteristics of the gas in operation S380. The concentration determination module 340 may interpret the separated gas signal as the product of the optical cross-sectional area and the concentration as shown in Equation (7), and the optical cross-sectional area may be determined by substituting the aerosol size, the wavelength of light, and the refractive and absorption constants called optical constants into the relational equation by Mie theory or Rayleigh theory.

The particle signal and the particle concentration determination will be described in detail with reference to FIGS. 4 to 8.

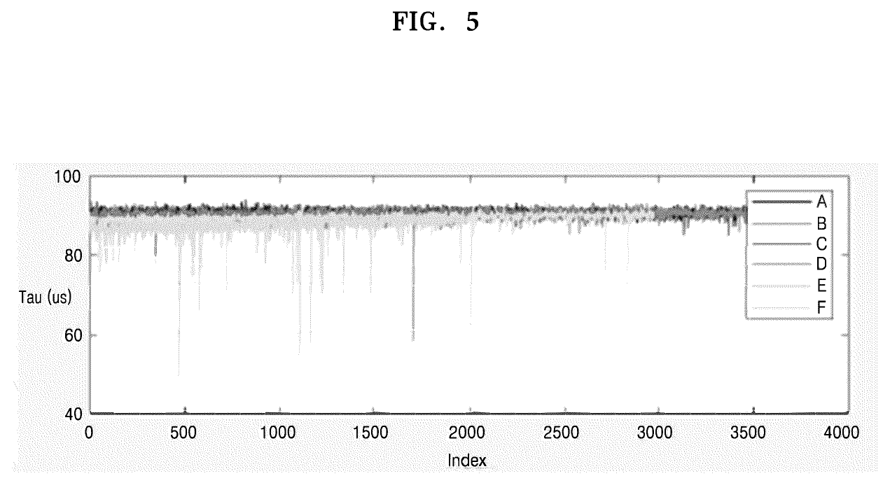

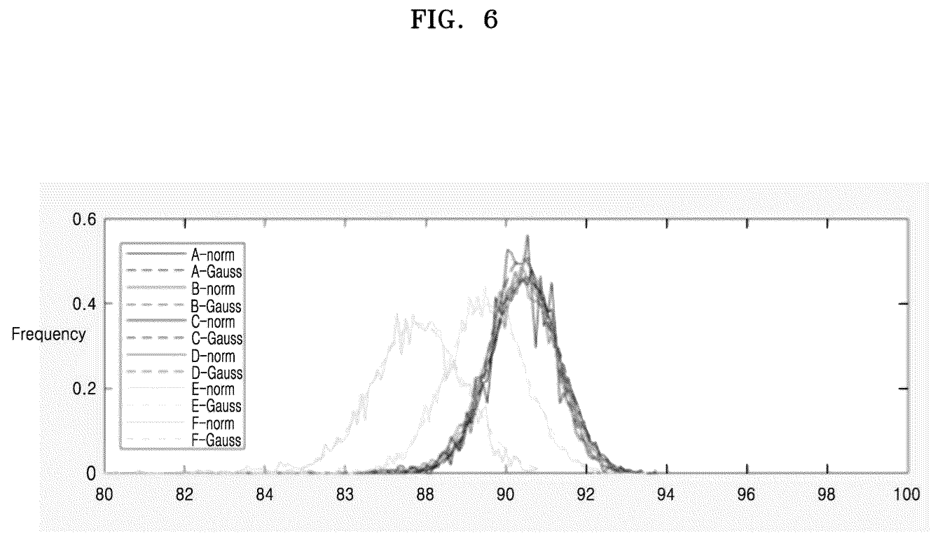

FIG. 4 is a flowchart illustrating an operation of determining a concentration of particles in an optical measurement method according to one or more embodiments. FIG. 5 is a graph illustrating an optical signal including a ring-down time value in an optical measurement device according to one or more embodiments. FIG. 6 is a graph illustrating a result of fitting a particle signal to a first distribution function indicating a frequency of a ring-down time value in an optical measurement method according to one or more embodiments.

Referring to FIG. 4, operation S390 of determining the particle concentration through the particle signal in FIG. 1 may include operation S392 of fitting the particle signal to a first distribution function indicating a frequency of an optical characteristic value (e.g., a ring-down time value), operation S394 of fitting the particle signal to a second distribution function indicating a cumulative frequency of an optical characteristic value (e.g., a ring-down time value), and operation S396 of analyzing a higher-order standard moment for the first distribution function. The operation S390 of determining the concentration of the particles through the particle signal may be performed through the concentration determination module 340 of FIG. 2.

Although FIG. 4 illustrates that the operation S394 of fitting to the second distribution function is performed prior to the operation S396 of analyzing the higher-order standard moment, embodiments are not limited thereto. For example, the operation of analyzing the higher-order standard moment (operations S396) may be performed before the operation of fitting to the second distribution function (operation S394), or the operation of analyzing the higher-order standard moment (operation S396) and the operation of fitting to the second distribution function (operation S394) may be performed simultaneously. In addition, the operation of fitting to the first distribution function (operation S392), the operation of fitting to the second distribution function (operation S394), and the operation of analyzing the higher-order standard moment (operation S396) may be performed substantially simultaneously by the operation process of the concentration determination module 340.

In one or more embodiments, the first distribution function may be a Gaussian distribution function indicating an optical characteristic value (e.g., a ring-down time value) and a frequency of the optical characteristic value (e.g., a frequency of data with a corresponding ring-down time value) with respect to the particle signal.

In one or more embodiments, the second distribution function may be a cumulative distribution function indicating an optical characteristic value (e.g., a ring-down time value) and a cumulative frequency of the optical characteristic value (e.g., a cumulative frequency of data with a corresponding ring-down time value) with respect to the particle signal.

FIG. 5 illustrates data represented as a ring-down time value (Y-axis, Tau (τ)) according to a time (X-axis, Index) of each particle signal of Sample A to Sample F after extracting each particle signal with respect to the Sample A, Sample B, Sample C, Sample D, Sample E, and Sample F. These may be data extracted from the detector 300 (FIG. 2) through the optical signal detection module 310 (FIG. 2), the electrical signal conversion module 320 (FIG. 2), and the electrical signal recording module 330 (FIG. 2).

For reference, samples A to F are samples corresponding to ISO Class 4 to ISO Class 9, respectively, and it may be understood that the particle concentration of sample A is the lowest and the particle concentration of sample F is the highest. As shown in FIG. 5, when a particle signal is represented as a ring-down time value (Y-axis, Tau (τ)) according to time (X-axis, Index) and a separate particle signal processing method is not applied, it may be difficult to distinguish Sample A to Sample F from one another as shown in FIG. 5. That is, since it is not easy to determine the concentration of samples A to F, the particle signal may become data discarded after being separated from the gas signal.

The optical measurement method according to one or more embodiments may include an operation S392 of fitting the particle signal separated from the gas signal to a first distribution function indicating the frequency of the ring-down time value τ.

FIG. 6 illustrates a general distribution curve (A-norm to F-norm) representing a frequency of a ring-down time value (τ) and a fitted first distribution function curve (A-Gauss to F-Gauss) with respect to the particle signal of each of Sample A to Sample F. As described above, the first distribution function may be fitted to the Gaussian distribution function.

An optical measurement method according to one or more embodiments may include determining the concentration of particles using first shape information about the first distribution function. For example, when the first distribution function is a Gaussian distribution function, the first shape information may include a mean (or median value) and/or a variance.

A median value of each of the first distribution function curves (A-Gauss to F-Gauss) of the sample A to the sample F shown in FIG. 6 may be obtained. The median value (τ_{0A}) of sample A is determined as 90.381 μs, the median value (τ_{0B}) of sample B is determined as 90.437 μs, the median value (Toc) of sample C is determined as 90.485 μs, the median value (τ_{0D}) of sample D is determined as 90.3894 μs, the median value (τ_{0E}) of sample E is determined as 89.4992 μs, and the median value (τ_{0F}) of sample F is determined as 87.8237 μs. That is, the higher the particle concentration, the less the median value tends to be. In operation S390 of determining the particle concentration, median values of the first distribution function curves A-Gauss to F-Gauss for each sample may be used.

In order to improve the resolution of particle concentration measurement, the optical measurement method according to one or more embodiments may include operation S396 of extracting a higher-order standard moment with respect to the first distribution function. In one or more embodiments, operation S390 of determining the particle concentration may be performed by extracting the variance, which is the secondary moment, the skewness, which is the tertiary moment, and the kurtosis, which is the quaternary moment, as well as the mean, which is the primary moment. That is, the first shape information about the first distribution function may include mean, variance, skewness, and kurtosis. In the first shape information, the mean may indicate a median value, the variance may indicate a width, the skewness may indicate asymmetry, and the kurtosis may indicate the thickness of the tail. Thus, the concentration of particles may be determined using the first shape information. For example, the greater the mean, the lower the particle concentration, and the less the variance, the lower the particle concentration may tend to be. The statistical methodology may be applied to determine the particle concentration.

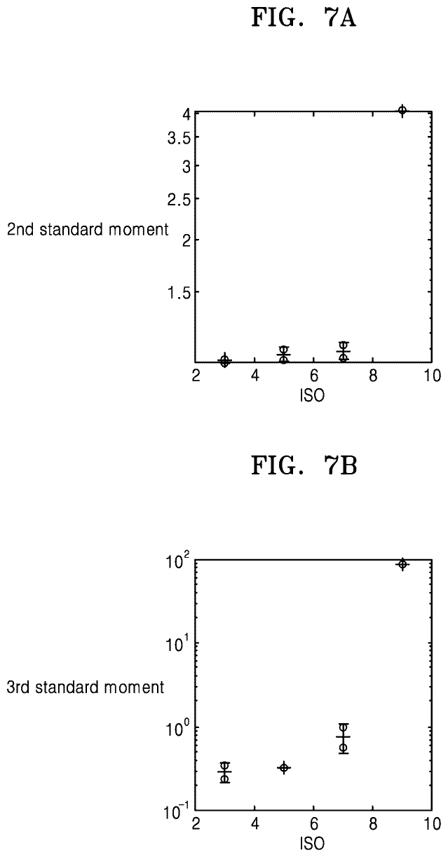

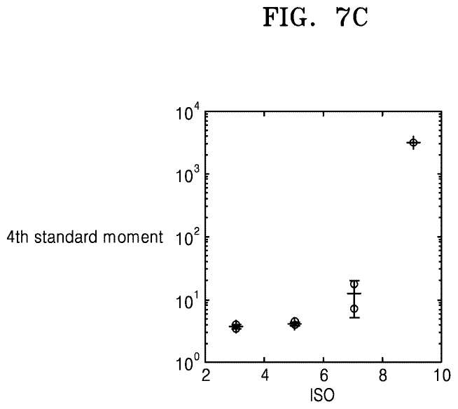

FIGS. 7A to 7C are graphs illustrating secondary to quaternary standard moments of a first distribution function extracted in an optical measurement method according to one or more embodiments. In particular, FIGS. 7A to 7C are graphs illustrating secondary to quaternary standard moments of a first distribution function extracted from samples of ISO Class 3, ISO Class 5, ISO Class 7, and ISO Class 9. FIG. 7A shows a secondary standard moment corresponding to the variance of the first distribution function, FIG. 7B shows a tertiary standard moment corresponding to the skewness of the first distribution function, and FIG. 7C shows a quaternary standard moment corresponding to the kurtosis of the first distribution function.

When looking at examples of the samples of ISO Class 5 and ISO Class 7 shown in FIGS. 7A to 7C, each of the samples of the ISO Class 5 and the ISO Class 7 may correspond to the sample B (ISO Class 5) and the sample D (ISO Class 7) of FIG. 6. The median value (τ_{0B}=90.4347 μs) of sample B in FIG. 6 is easily distinguished from the median value (τ_{0E}=89.4992 μs) of sample E and the median value (τ_{0F}=870.8237 μs) of sample F, both of which have high contamination, but has a value similar to the median value (τ_{0D}=90.3894 μs) of sample D with relatively low contamination.

Referring back to FIGS. 7A to 7C, the secondary standard moment of ISO Class 5 and the secondary standard moment of ISO Class 7 shown in FIG. 7A are both similar. However, the tertiary standard moment of ISO Class 5 shown in FIG. 7B may be distinguished from the tertiary standard moment of ISO Class 7, and the quaternary standard moment of ISO Class 5 and the quaternary standard moment of ISO Class 7 shown in FIG. 7C may be distinguished from each other. In this way, the resolution for determining the concentration of the particles may be increased by extracting the higher-order standard moment for the first distribution function. In particular, it may be easy to compare, with each other, samples having a relatively low level of contamination.

According to one or more embodiments, in operation S390 of determining the particle concentration, the particle concentration may be determined according to a combination of the mean, variance, skewness, and kurtosis as well as using determined values of each of the mean, variance, skewness, and kurtosis included in the first shape information. That is, the concentration of particles may be determined based on a combination of the primary moment, the secondary moment, the tertiary moment, and the quaternary moment of the first distribution function.

In the optical measurement method according to one or more embodiments, operation S390 of determining the particle concentration may include operation S394 of fitting the particle signal to a second distribution function indicating the cumulative frequency of an optical characteristic value (e.g., a ring-down time value), and the particle concentration may be determined by using the second shape information about the second distribution function together with the first shape information about the first distribution function.

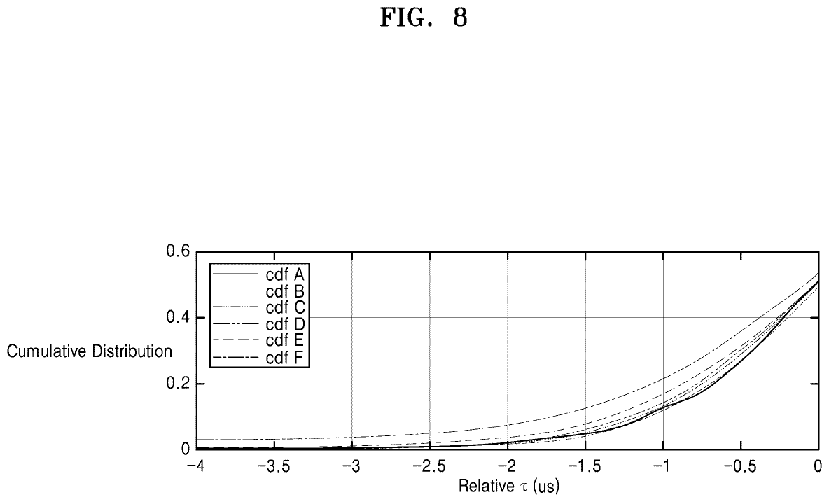

FIG. 8 is a graph illustrating a result of fitting a particle signal to a second distribution function indicating a cumulative frequency of an optical characteristic value in an optical measurement method according to one or more embodiments. The second distribution function may be a cumulative distribution function. The graph of FIG. 8 shows the particle signals of respective samples A to F shown in FIG. 6 as a cumulative distribution function curve showing the cumulative frequency of the ring-down time value (τ).

The second shape information about the second distribution function, which is a cumulative distribution function (shown as CDF in FIG. 8), reflects information about the tail region of the distribution of the ring-down time value so that the overall difference in the curved shape may be clarified. The second distribution function as shown in FIG. 8 may facilitate the distinction between samples having relatively low levels of contamination (e.g., ISO Class 4 and ISO Class 5), and may improve particle concentration measurement sensitivity.

In the optical measurement method according to one or more embodiments, the obtained optical signal may be separated into a gas signal and a particle signal, the gas signal may be converted into a gas concentration using an equation such as a CRDS equation, the particle signal may be fitted to a distribution function for the ring-down time value, and the particle concentration may be determined using the shape information about the distribution function. The distribution function may include a first distribution function fitted to a Gaussian distribution function and a second distribution function fitted to a cumulative distribution function. The first distribution function may include information about the mean and/or variance of the Gaussian distribution, and the second distribution function may include information about a tail region of the distribution of the ring-down time value. In addition, a higher-order standard moment may be extracted for the first distribution function to include information about variance, skewness, and/or kurtosis of the Gaussian distribution. That is, the first shape information about the first distribution function may include mean, variance, skewness, and kurtosis, and the second shape information about the second distribution function may include information about an outlier corresponding to the tail region of the distribution. The concentration of particles may be determined using the first shape information about the first distribution function and the second shape information about the second distribution function.

The samples of FIGS. 5 to 8 have been described together with the actual particle concentration to explain the difference of the first shape information and the difference of the second shape information according to the difference in particle concentration. According to one or more embodiments, the concentration determination module 340 may determine the concentration of particles by comparing the first shape information and the second shape information with previously collected data in a database.

As described above, by determining the gas concentration and particle concentration together in one optical signal, the mechanism by which gas and particles are converted to each other may be identified, thereby enabling thermodynamic and reaction rate chemical reaction studies on the reaction and conversion between particles and gases. In addition, the optical measurement device according to one or more embodiments may simultaneously function as a gas concentration measuring instrument and a particle counter, thereby simplifying multiple analysis facilities for determining gas and particle concentrations.

Embodiments herein have mainly been described in the case where the optical characteristic value included in the optical signal is the ring-down time value. In one or more embodiments, when an optical facility other than CRDS is used, the optical characteristic value may vary. For example, the optical characteristic value may correspond to a sum of light emitted from cavity, a phase difference of light, or the like. Alternatively, the sum of light and the phase difference of light may be substituted with a ring-down time value using predetermined equations.

As used in connection with various embodiments of the disclosure, the term “module” may include a unit implemented in hardware, software, or firmware, and may interchangeably be used with other terms, for example, logic, logic block, part, or circuitry. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software including one or more instructions that are stored in a storage medium that is readable by a machine. For example, a processor of the machine may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

At least one of the devices, units, components, modules, units, or the like represented by a block or an equivalent indication in the above embodiments including, but not limited to, FIGS. 2, may be physically implemented by analog and/or digital circuits including one or more of a logic gate, an integrated circuit, a microprocessor, a microcontroller, a memory circuit, a passive electronic component, an active electronic component, an optical component, and the like, and may also be implemented by or driven by software and/or firmware (configured to perform the functions or operations described herein).

Each of the embodiments provided in the above description is not excluded from being associated with one or more features of another example or another embodiment also provided herein or not provided herein but consistent with the disclosure.

While the disclosure has been particularly shown and described with reference to embodiments thereof, it will be understood that various changes in form and details may be made therein without departing from the spirit and scope of the following claims.

What is claimed:

1. An optical measurement method comprising:
emitting light to an optical beam path cell comprising a first mirror and a second mirror that faces the first mirror;
forming an optical beam path by reflecting the light between the first mirror and the second mirror;
obtaining an optical signal including an optical characteristic value based on an interaction of the light with a sample in the optical beam path;
separating the optical signal into a gas signal and a particle signal; and
determining a concentration of particles in the sample based on the particle signal,
wherein the determining of the concentration of the particles comprises fitting the particle signal into a first distribution function indicating the optical characteristic value and a frequency of the optical characteristic value, and
wherein the concentration of particles is determined based on first shape information about the first distribution function.

2. The optical measurement method of claim 1, wherein the optical characteristic value comprises a ring-down time value.

3. The optical measurement method of claim 1, wherein the first distribution function comprises a Gaussian distribution function.

4. The optical measurement method of claim 1, wherein the determining of the concentration of the particles further comprises fitting the particle signal to a second distribution function indicating the optical characteristic value and a cumulative frequency of the optical characteristic value, and
wherein the concentration of the particles is determined based on the first shape information about the first distribution function and second shape information about the second distribution function.

5. The optical measurement method of claim 1, wherein the determining of the concentration of the particles further comprises extracting a higher-order standard moment corresponding to the first distribution function.

6. The optical measurement method of claim 5, wherein the first shape information comprises at least one of a mean, a variance, a skewness, and a kurtosis.

7. The optical measurement method of claim 6, wherein the first shape information comprises a combination of the mean, the variance, the skewness, and the kurtosis, and
wherein the concentration of the particles is determined based on the combination of the mean, the variance, the skewness, and the kurtosis.

8. The optical measurement method of claim 1, further comprising determining a concentration of gas in the sample from the gas signal based on an optical characteristic of the gas.

9. The optical measurement method of claim 1, wherein the optical measurement method is performed based on at least one of cavity ring-down spectroscopy (CRDS), integrated cavity output spectroscopy (ICOS), cavity enhanced abstraction spectroscopy (CEAS), and cavity attenuated phase shift spectroscopy (CAPS).

10. The optical measurement method of claim 1, wherein the optical beam path is an open path exposed to an environment.

11. The optical measurement method of claim 1, wherein the light emitted to the optical beam path cell has a preset wavelength based on a type of particle, and
wherein the light of the preset wavelength comprises at least one of ultraviolet (UV) light, visible light, infrared (mid-IR) light, near-infrared (near-IR) light, far-infrared (far-IR) light, submillimeter (sub-mm), and terahertz (Thz) light.

12. An optical measurement device comprising:
a light source configured to emit light;
an optical beam path cell comprising a first mirror and a second mirror configured to form an optical beam path by reflecting the light emitted from the light source; and
a detector configured to obtain an optical signal based on an interaction of the light with an aerosol sample in the optical beam path, and determine a concentration of particles and a concentration of gas in the aerosol sample from the optical signal,
wherein the detector comprises a concentration determination module configured to fit a particle signal extracted from the optical signal into a Gaussian distribution function representing a ring-down time value and a frequency of the ring-down time value; and
determine the concentration of the particles based on first shape information about the Gaussian distribution function.

13. The optical measurement device of claim 12, wherein the concentration determination module is further configured to extract a higher-order standard moment for the Gaussian distribution function, and
wherein the first shape information includes mean, variance, skewness, and kurtosis.

14. The optical measurement device of claim 13, wherein the concentration determination module is further configured to:
fit the particle signal to a cumulative distribution function indicating the ring-down time value and a cumulative frequency of the ring-down time value; and
determine the concentration of the particles based on second shape information about the cumulative distribution function.

15. The optical measurement device of claim 14, wherein the concentration determination module is further configured to determine the concentration of the particles by comparing the first shape information and the second shape information with a database storing previously collected data.

16. The optical measurement device of claim 12, wherein the optical beam path is an open path exposed to an environment.

17. The optical measurement device of claim 12, wherein the optical measurement device uses multi-pass spectroscopy, and
wherein the multi-pass spectroscopy is one of cavity ring-down spectroscopy (CRDS), integrated cavity output spectroscopy (ICOS), cavity enhanced abstraction spectroscopy (CEAS), and cavity attenuated phase shift spectroscopy (CAPS).

18. An optical measurement method comprising:
emitting light to an optical beam path cell comprising a first mirror and a second mirror that faces the first mirror;
forming an optical beam path by reflecting the light between the first mirror and the second mirror;
measuring an optical signal from an aerosol sample in the optical beam path;
separating the optical signal into a gas signal and a particle signal; and
determining a concentration of particles and a concentration of gas in the aerosol sample based on the particle signal,
wherein the determining of the concentration of the particles comprises fitting the particle signal into a Gaussian distribution function indicating a ring-down time value and a frequency of the ring-down time value, and
wherein the concentration of the particles is determined based on a mean, a variance, a skewness, and a kurtosis of the Gaussian distribution function.

19. The optical measurement method of claim 18, wherein the determining of the concentration of the particles further comprises fitting the particle signal to a cumulative distribution function indicating the ring-down time value and a cumulative frequency of the ring-down time value.

20. The optical measurement method of claim 19, wherein the separating of the optical signal into the gas signal and the particle signal is performed based on a magnitude of the ring-down time value in the optical signal.
